(19) United States
(12) Patent Application Publication
(10) Pub. No.: US 2019/0159956 A1
(43) Pub. Date: May 30, 2019

(54) METHOD AND APPARATUS FOR TREATING DIPLOPIA AND CONVERGENCE INSUFFICIENCY DISORDER

(71) Applicant: Amblyotech Inc., Boston, MA (US)

(72) Inventor: Joseph Koziak, Boston, MA (US)

(21) Appl. No.: 16/199,017

(22) Filed: Nov. 23, 2018

Related U.S. Application Data
(60) Provisional application No. 62/590,472, filed on Nov. 24, 2017.

(51) Int. Cl. A61H 5/00; A61B 3/08

(52) U.S. Cl. CPC A61H 5/005; H04N 2013/0074; A61B 3/08

(57) ABSTRACT

A method of treating diplopia or convergence insufficiency disorder in a patient; it includes providing a patient having a condition of diplopia or convergence insufficiency disorder with an image pair configured to present a first image to a first weaker eye of the patient and a second image to a second dominant eye of the patient; obtaining performance information of the patient when the patient performs a task requiring the perceiving of at least the information content of the first image; and adjusting, based on the performance information, the at least one image parameter such that the difference in perceptibility of the information content of the first image and the information content of the second image is reduced.

The present patent application claims priority from U.S. provisional patent application No. 62/590,472 filed on Nov. 24, 2017 that is incorporated herein by reference.

### TECHNICAL FIELD

The present application relates to a method and apparatus for treating a patient with diplopia and/or with convergence insufficiency disorder.

### BACKGROUND

Diplopia is the simultaneous perception of two images of a single object that may be displaced horizontally, vertically, diagonally or rotationally with respect to one another. Diplopia may be the result of impaired function of the extraocular muscles. Diplopia is sometimes present in patients suffering from other ocular disorders, for example amblyopia, where the weak eye may be left to wander.

Convergence insufficiency disorder is a binocular vision disorder in which at least one eye has a tendency of drifting outward when reading or doing work close up. Diplopia may result when the eye drifts out.

Hess et al. (Hess R F, Mansouri B, Thompson B. A new binocular approach to the treatment of amblyopia in adults well beyond the critical period of visual development. Restor Neurol Neurosci 2010; 28:793-802) reported a binocular paradigm for treatment of amblyopia consisting of laboratory-based perceptual learning sessions. In these sessions, dichoptic motion coherence thresholds were measured, and contrast levels in the fellow eye were adjusted to optimize combination of visual information from both eyes and overcome suppression of the amblyopic eye. Nine adults (aged 24 to 49 years) were treated, with amblyopic eye visual acuity ranging from 20/40 to 20/400. Treatment resulted in significantly improved amblyopic eye visual acuity (P<0.008) and stereoacuity (P=0.012), despite 4 of 9 (44%) subjects previously being treated with patching. Knox et al (Knox P J, Simmers A J, Gray L S, Cleary M. An exploratory study: prolonged periods of binocular stimulation can provide an effective treatment for childhood amblyopia. Invest Ophthalmol Vis Sci 2012; 53:817-824) studied a similar paradigm with a binocular Tetris game using an in-office, head-mounted display over five 1-hour treatment sessions. Contrast was adjusted to equalize input from each eye. Fourteen children (aged 6 to 14 years) with previously treated amblyopia (patching) were included in the study, with amblyopic eye visual acuity ranging from 20/32 to 20/160. Following treatment mean amblyopic eye visual acuity had improved significantly (P=0.0001) despite previous treatment with patching. Six of the 14 children improved 0.1 log MAR or more and stereoacuity also improved significantly (P=0.02). In another recent study published in 2013, Li et al. (Li J, Thompson B, Deng D, Chan L Y, Yu M, Hess R F. Dichoptic training enables the adult amblyopic brain to learn. Curr Biol 2013; 23:R308-309) used the Tetris video game, presented via head-mounted video goggles, one hour per day for two weeks of in-office sessions. Eighteen adults were treated in a crossover design comparing monocular game play with dichoptic game play, using adjustment of contrast to allow for binocular combination. Following treatment, dichoptic game play was found to significantly improve stereoacuity, visual acuity, and contrast balance between fellow and amblyopic eye compared with monocular game play. In these prior studies by Hess and Knox, of note is the finding that visual acuity was found to improve despite prior treatment of amblyopia (44% of cases in Hess study and 100% in Knox study). Regarding amblyopia mechanism (strabismic, anisometropic, or combined), there was no evidence for one type of amblyopia to respond better with binocular amblyopia treatment.

These previous studies of binocular treatment have relied on in-office sessions to perform the respective binocular treatment paradigms, but Hess' group has recently adapted the binocular approach to a game platform on an iPod30, 31 and now on an iPad. Using an iPod or iPad provides greater flexibility to the implementation of binocular treatment.

Li and Birch et al. (Li S, Subramanian V, To L, et al. Binocular iPad treatment for amblyopia. Invest Ophthalmol Vis Sci 2013; 54:4981 (ARVO meeting abstract) studied treating amblyopia with dichoptic iPad games, using red-green anaglyphic glasses, for 4 hours/week for 4 weeks, and reported a mean improvement from 0.47+0.19 log MAR at baseline to 0.39+0.19 log MAR (p<0.001) after 4 weeks of binocular treatment in 50 children age 5 to 11 years. They found no significant mean improvement in visual acuity of 25 children assigned to sham treatment. Some children in each group also were treated with monocular patching, at a different time of day, at the discretion of the treating physician. Nevertheless, children treated with binocular games alone improved a mean of 0.08+0.07 log MAR. Although 4 games were available to each child, most children played the Tetris game or the balloon game.

In a subsequent study in younger children (3 to <7 years), Birch et al (Birch E E, Li S, Jost R M, et al. Binocular iPad treatment for amblyopia in preschool children. J AAPOS 2014 (AAPOS meeting abstract)) reported no change in visual acuity with sham iPad games for 4 hours/week for 4 weeks (n=5), but an improvement from 0.43+0.2 log MAR to 0.34+0.2 log MAR in 45 children treated with dichoptic iPad games for 4 hours/week for 4 weeks (p<0.001). Children who played the games 8 or more hours total playing time over the 4-week treatment period had significantly greater improvement that those who played 0-4 hours (0.14+0.11 log MAR vs 0.01+0.04 log MAR (p=0.0001). Although these children were allowed to patch during the study (at the discretion of the treating physician), those who played >8 hours and had no patching showed an improvement of 0.14+0.16 log MAR at 4 weeks. Although 4 different games were available to each child, most children played the Tetris game or the balloon game (E. Birch, personal communication).

These studies provide “proof of concept” for the effectiveness of binocular treatment in amblyopia in children and adults, and the studies demonstrate feasibility of using the iPad format, wearing red-green anaglyphic glasses, for implementing binocular treatment in a pediatric population.

It was however postulated that the binocular treatment described above, as developed by Hess's group, would result in increased symptoms of diplopia and/or convergence insufficiency disorder.

The disclosure in this Background section does not constitute an admission of applicable prior art.

### SUMMARY

The present disclosure relates to the treatment and diminishing of diplopia and convergence insufficiency disorder (CID) in patients, whether the cause of diplopia and/or CID is, for example, amblyopia, muscular dystrophy, or another condition, disorder or illness of the patient.

It has been discovered that an apparatus providing a first image perceivable by a first eye of a patient and a second image perceivable by a second eye of the patient, where the information content between the first perceivable image and the second perceivable image is different, and where image parameters (of the image(s) of the image pair) may vary such that the information content in one perceivable image is more perceivable than in the other, assists with the treatment of diplopia and/or CID, and lowers the risk of presence of diplopia and/or CID for the patient (independent of the cause of diplopia and/or CID). The patient is asked to perform a task using the information content from the image perceived by the weak eye, or from both perceivable images, requiring that information received by both eyes (corresponding to the two perceivable images) be processed by the patient's brain. The performance of a task for a given period (or of different tasks using the information content from both perceivable images) may result in reducing the presence of diplopia and/or CID as experienced by the patient, and/or treat diplopia and/or CID.

By information content, it is meant the visual components of the images, such as the objects or items appearing in the image. For example, in the case of a computer game where the objective is to collect gold coins, e.g., the characters, platforms on which the characters may mount and the gold coins are related to the information content of the image. In the case of an image pair (e.g. the first image as perceivable by a first eye and a second image as perceivable by a second eye), the information content in one image may be different from the image content in the other. An image pair is at least one image that is adapted to present a first perceivable image to a left eye and a second perceivable image to a right eye, where the first perceivable image is configured to present to the left eye information content that is different from the information content that the second perceivable image is configured to present. In some examples, the image pair may be one image that is adapted to be viewed using anaglyphic glasses (e.g. red-green glasses), where some information content is perceivable by the left eye, and other information content is perceivable by the right eye when the patient wears the anaglyphic glasses. In other examples, the image pair may be two images, a first image for the right eye and a second image for the left eye, where the information content perceivable by the right eye as presented on the first image is at least partially different from the information content perceivable by the left eye as presented on the second image. In the example of the game described above, the gold coins may be perceivable by one eye (i.e. the first perceivable image), where the characters and platforms may be perceivable by the second eye (i.e. the second perceivable image). In some examples, the background of the image pair, or some of the elements of the image, may be common to both perceivable images, where each of the patient's eyes picks up on the common information content (e.g. in the case of a videogame, the background may be a common landscape present in both images; in some examples, the platforms on which the characters find support may be present in both images, etc.)

In some examples, the image pair may be, e.g., an image stream (e.g. a video, an interactive stream of images of a computer game, etc.), a static image, a sequence of static images, etc. In some examples, the image pair may be presented in a virtual reality environment, an augment reality environment or in an enhanced reality environment (e.g. where the physical world may serve as a landscape consisting of common information content for the first perceived image and the second perceived image, and additional information content is added to the image pair such as the virtual information content presented to a left eye that is different from virtual information content presented to a right eye).

The image parameters relate to, for example, the brightness, luminance, contrast, hue, resolution, filtering, etc., of the image. The image parameters may be adjusted as the patient performs the given task, or may be adjusted at the beginning of the task. In some examples, only certain portions of the image may be adjusted by the image parameters (e.g. blobs or quadrants of the image). In some examples, the image parameters may be adjusting the quantity of information content in an image or both images (e.g. the number of objects appearing in one image).

By adjusting the image parameters, the information content of a first perceivable image may be more perceivable to the eye corresponding to that image than the information content of the second perceivable image perceivable by the other corresponding eye. In some examples, where the patient suffers from diplopia and/or CID, the image with a first portion of information, that may be more perceivable, is presented to the weak eye (e.g. the wandering eye), and the image with a second portion of information, that may be less perceivable, is presented to the dominant eye. As such, the brain begins to process the image being received by the weak eye and its information content. The image parameters may be adjusted over time as the weak eye is strengthened and the presence of diplopia and/or CID diminishes, such that the perceptibility of the information content between both images becomes more or less equal as the patient's condition is treated.

A broad aspect is a method of treating diplopia in a patient. The method includes providing a patient having a condition of diplopia with an image pair configured to present a first image to a first weaker eye of the patient and a second image to a second dominant eye of the patient, wherein information content of the first image perceivable by the first eye is different from information content of the second image perceivable by the second eye, and wherein the perceptibility of the information content of the first image is augmented in comparison to the perceptibility of the information content of the second image as a result of a difference in at least one image parameter of the first image and the second image. The method includes obtaining performance information of the patient when the patient performs a task requiring the perceiving of at least the information content of the first image. The method includes adjusting, based on the performance information, when the performance information is indicative of an improvement of the patient's diplopia symptoms, the at least one image parameter such that the difference in perceptibility of the information content of the first image and the information content of the second image is reduced.

In some embodiments, the image pair may be generated from a single image source configured to be used with anaglyphic glasses, wherein the patient wearing the anaglyphic glasses may result in the presenting of the first image to the first weaker eye of the patient and the second image to the second dominant eye of the patient.

In some embodiments, the image pair may include a first image source for generating the first image presented to the first eye and a second image source for generating the second image presented to the second eye.

In some embodiments, the image pair may be generated from an image source configured to generate an image stream.

In some embodiments, the at least one image parameter may be the number of objects appearing in the first image and the number of objects appearing in the second image.

In some embodiments, the at least parameter may be the contrast of the first image and the second image.

In some embodiments, the difference in perceptibility may affect only a portion of at least one of the first image and the second image.

In some embodiments, the task may be established within the context of a video game.

In some embodiments, the image pair may be provided while the patient is wearing an augmented reality headset.

In some embodiments, the information content of the first image may be layered over a live stream of images generated from a camera.

In some embodiments, the at least one image parameter may affect objects appearing in the live stream of images generated from a camera.

In some embodiments, the image pair may be provided while the patient is wearing a virtual reality headset or virtual reality glasses.

In some embodiments, the information content of the first image may be layered over a live stream of images generated from a camera.

In some embodiments, the at least one image parameter may affect objects appearing in the live stream of images generated from a camera.

In some embodiments, the patient may have diplopia.

In some embodiments, the patient may have convergence insufficiency disorder.

### BRIEF DESCRIPTION OF THE DRAWINGS

The invention will be better understood by way of the following detailed description of embodiments of the invention with reference to the appended drawings, in which:

FIG. 1 is a graph illustrating a distribution of reports of diplopia as a function of hours of gameplay over subjects between the ages of 13 years old and less than 17 years old as reported by the subjects;

FIG. 2 is a graph illustrating a distribution of reports of diplopia as a function of hours of gameplay over subjects between the ages of 5 years old and less than 13 years old as reported by the subjects;

FIG. 3 is a graph illustrating a distribution of reports of diplopia as a function of hours of gameplay over subjects between the ages of 13 years old and less than 17 years old as reported by the parents of subjects;

FIG. 4 is a graph illustrating a distribution of reports of diplopia as a function of hours of gameplay over subjects between the ages of 5 years old and less than 13 years old as reported by the parents of the subjects;

FIG. 5 is a graph illustrating a distribution of reports of diplopia as a function of hours of gameplay over subjects of all age groups as reported by the subjects;

FIG. 6 is a graph illustrating a distribution of reports of diplopia as a function of hours of gameplay over subjects of all age groups as reported by the parents of the subjects;

FIG. 7 is a block diagram of an exemplary apparatus for treating diplopia and/or CID; and

FIG. 8 is a flowchart diagram of an exemplary method of treating diplopia and/or CID.

### DETAILED DESCRIPTION

The present disclosure relates to an apparatus and methods for treating diplopia and/or CID. The disclosure pertains to training the weak eye (e.g. wandering eye) to function along with the dominant eye. The training includes presenting a first image to a first eye and a complementary second image to the second eye (i.e. image pairs). The patient is asked, based on the image pairs, to perform a task. Information content contained in at least the image presented to the weak eye is required to perform the task. If the patient is not picking up on the information content presented to the weak eye, the patient cannot complete the task. The ability of the patient to perform the task is an indication of the patient's processing the information presented to the weak eye. If the patient is not capable of performing the task, and is therefore not picking up on the information presented to the weak eye, then image parameters of the first image and/or the second image may be adjusted. For example, contrast and/or luminance may be adjusted such that the information content of the image presented to the weak eye is sharper and/or more vivid than the information content presented to the dominant eye. The adjustment may be furthered until the patient is picking up on the information content presented to both the weak eye and the dominant eye (e.g. both the first image and the second image). At these image parameters, the patient is then asked to carry out the task.

The physician may periodically adjust the image parameters as the patient comfortably completes the task over time, indicative of strengthening of the weak eye, such that both the perceived first image and the perceived second image have increasingly similar properties. This adjustment may be continued until both the perceived first image and the second image have identical image parameters. If the patient is capable of performing the tasks successfully when both images have identical parameters, this is indicative of the patient having regained function of the weak eye.

It will be understood that in the present disclosure, what is meant by a first image and a second image is that the first eye of the patient is perceiving an image that is different from the image perceived by the second eye of the patient. However, in some examples, this may not mean that an image (e.g. on a first screen) is presented to a first eye and a second distinct image (e.g. on a separate screen) is presented to the second eye. A single screen presenting a single image may be viewed by both eyes (e.g. on a handheld device). However, the image appearing on the screen may be adapted to be viewed anaglyphically (e.g. where the patient may be wearing anaglyphic glasses). In this example, the result is that the patient is perceiving with the first eye an image that is different from the image perceived by the second eye due to the properties of the image appearing on the screen and the anaglyphic glasses.

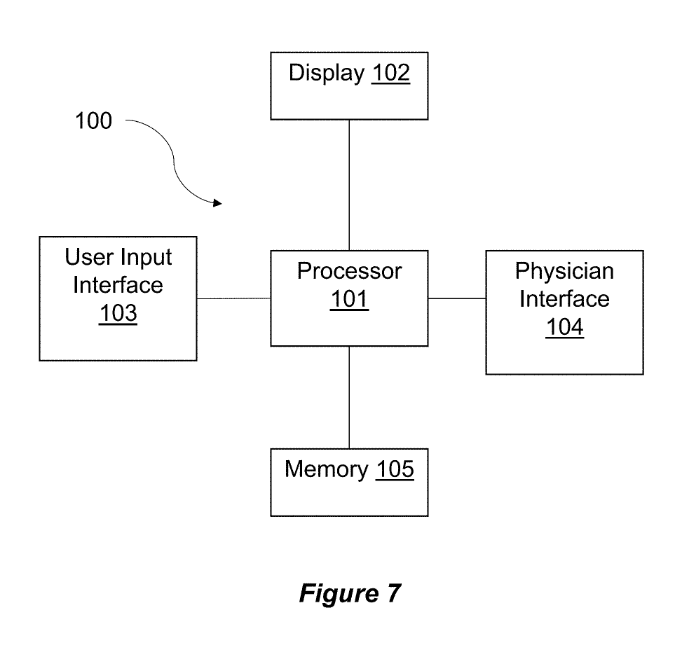

Reference is made to FIG. 7, illustrating an exemplary apparatus 100 for treating diplopia and/or CID.

The apparatus 100 has a processor 101, a user input interface 103, a memory 105 and a display 102. The apparatus 100 may also have a physician interface 104.

The memory 105 may contain program code for execution by the processor 101. Therefore, the memory 105 stores program instructions and data used by the processor 101. The computer readable memory 105, though shown as unitary for simplicity in the present example, may comprise multiple memory modules and/or cashing. In particular, it may comprise several layers of memory such as a hard drive, external drive (e.g. SD card storage) or the like and a faster and smaller RAM module. The RAM module may store data and/or program code currently being, recently being or soon to be processed by the processor 101 as well as cache data and/or program code from a hard drive.

The processor 101 is a general-purpose programmable processor. In this example, the processor 101 is shown as being unitary, but the processor may also be multicore, or distributed (e.g. a multi-processor). The processor 101 may be a micro-processor.

The user input interface 103 is an interface that allows the user to provide specific input, such as buttons to allow a user to play a game. For instance, the user input interface 103 may be a keyboard, a joystick, a controller, a touchpad, a microphone combined with a voice processor, a movement detector, etc. In some examples, the user input interface 103 may also provide for an option for the user to control the image parameters. In other examples, the image parameters may be controlled by a supervising physician.

In some examples where the user input interface 103 includes a microphone combined with a voice processor, the voice processor may carry out the commands pronounced by the patient. For instance, the apparatus 100 may be running with the Alexa application program, where Alexa may be configured to adjust certain parameters of the image pairs presented to the patient as a result of received input, or, e.g., transmit data to the supervising physician in response to a verbal request made by the patient.

In some examples, the apparatus 100 has a physician interface 104 configured to receive input from a medical practitioner or supervising physician. In some embodiments, the physician may control certain of the image parameters using the physician interface 104. In some embodiments, the physician interface 104 may also be configured to transmit information to the physician (e.g. via a wired or wireless connection) regarding, e.g., the patient's performance of the task, such as the patient's results, the settings of the apparatus 100, the game that is being played, comments provided by the patient, etc. In some examples, the physician interface 104 may be a transceiver, a transmitter and/or a receiver.

In some examples, the memory 105 stores the program code for the exercises and tasks to be carried out by the patient (e.g. the game). The program code may also include the instructions to generate the two images for a corresponding task.

The display 102 is a display that is used to present an image pair (i.e. a first image with different information content than that of the second image), where the first image is configured to be presented to a first eye of the patient; and the second image is configured to be presented to a second eye of the patient). In some examples, the difference in information content may be achieved between both images by using anaglyphic glasses (using the same image, but where some of the objects are configured to only appear to one eye, and some of the features are configured to only be perceived by the other eye), or by generating two distinct images, each with different information content. The display 102 may be, in some examples, a virtual reality headset, a headset display, augmented reality glasses such as Vuzic Blade AR Glasses, the screen of a portable computing device such as a tablet or smartphone, a desktop display, a television set, etc. The display 102 may have a wired connection to the processor 101.

In some examples, the display 102 may be adapted to be viewed using anaglyphic glasses.

The memory 105 and the processor 101 may have a BUS connection. The user input interface 103 and the physician interface 104 may be connected to the processor via a wired connection.

The apparatus 100 may be used to treat a patient with diplopia or CID.

The patient is provided with the apparatus 100. The apparatus 100 generates an image pair to be perceived visually by the patient, where each of the perceivable images provides each of the eyes with different information content with respect to one another. In one example, the image parameters are adjusted in at least one image such that the image content to be perceived by the weak eye is more perceivable than the image content to be perceived by the strong eye (e.g. by adjusting the contrast, the brightness of one image). The image parameters may be adjusted until the patient processes the information content from both images. In one example, this adjustment of parameters may be performed during a calibration phase. In one example, the image parameters may also be adjusted as the patient is performing the given task.

The patient's ability to perform the task provides an indication that the images received by both eyes are being processed by the brain. Image parameters may be adjusted throughout the course of treatment, and as the patient's vision improves. For instance, patients with CID may notice that the weak eye has less of a tendency to wander. Patients suffering from diplopia may notice that the diplopia-related symptoms, double-vision, begin to fade or not to present themselves during the course of treatment. Therefore, as the patient continues to perform the tasks during treatment, less will the symptoms of diplopia present themselves.

It will be understood that in some examples, the apparatus 100 may be, for example, a smartphone, tablet, or computer, having stored in memory and/or running an application program configured to present an image pair as described herein (e.g. the application program may be played over the Internet, accessible via, e.g., a webpage, or downloaded and stored in memory on the computer device).

Examples of tasks to be performed may be in the context of a game. For instance, a first game may be to click on the bad monsters and avoid the good monsters. The bad monsters perceivable in a first image may be configured to only be perceived by the weak eye, where the good monsters perceivable in a second image may be configured to only be perceived by the strong eye. The patient's brain has to process the image perceived by the weak eye showing the bad monsters to complete the task of the game. Such may be achieved by adjusting the image parameters as explained herein.

In another example, the game may be one to jump over moving obstacles that are on a track as the obstacles approach a visible controllable character as the game progresses. The character may be perceivable by the strong eye, where the obstacles may be perceivable by the weak eye. The patient has to process the information content present on both images in order to perform the task of the game.

Method of Treating Diplopia and/or CID:

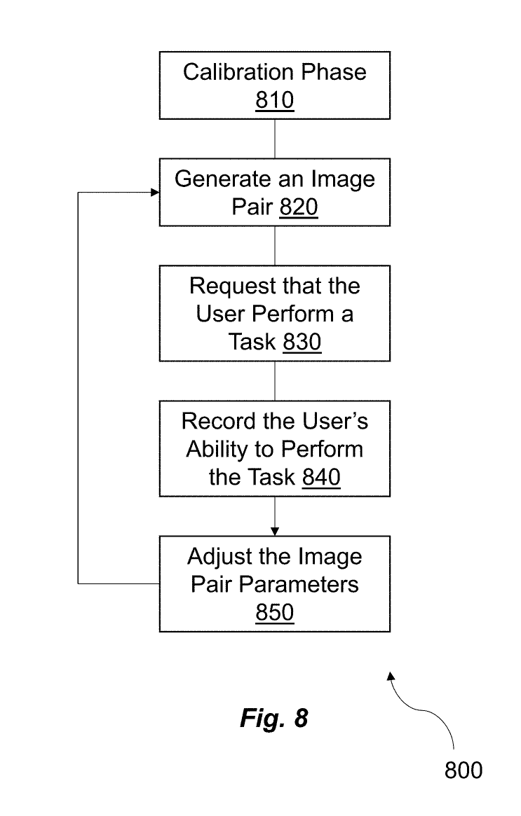

Reference is now made to FIG. 8, illustrating an exemplary method 800 of treating diplopia and/or CID of a patient. The exemplary method 800 may employ an exemplary apparatus 100 as described herein. For the purposes of illustration, reference is made, when describing exemplary method 800, to exemplary apparatus 100. However, it will be understood that an apparatus other than exemplary apparatus 100 may be used.

The apparatus 100 is first calibrated at step 810 in order to set the image parameters of the image pair presented to the patient. The image parameters are adjusted based on the extent of the visual condition of the patient. For instance, there may be, during the calibration phase, an exercise requesting that the patient position a first arrow, apparent in one of the perceived images, vis-à-vis a second arrow, apparent in the second perceived image. The patient or the supervising physician may adjust the image parameters until both arrows are perceived by the patient. In some examples, a program code may be executed by the processor of the apparatus to gradually adjust the image parameters until both arrows are perceptible (e.g. perceptibility indicated from input received from the user). For example, the patient may then indicate, for instance, by using the input interface, that both arrows can be perceived. At this stage, the image parameters may be set. The image parameters may be adjusted for one image, or for both images.

An image pair (e.g. which may be an image stream configured such that a first image is perceived by a first eye and a second image is perceived by a second eye) is generated at step 820 with the image parameters set in accordance with those established during the calibration step 810. It will be understood that, in some examples, the image parameters may be set by applying a filter (e.g. an optical filter) over an image, or portions of the image.

For instance, in some examples, the image pair may be provided when the patient is wearing an augmented reality headset. In these examples, an image stream is being taken of a real-life event, where, for instance, certain objects in the image stream are either altered to be removed or some objects added, where certain objects are perceivable by one eye and other objects are perceivable by the other eye. In some embodiments, the information content of the first image may be layered over a live stream of images generated from a camera (e.g. computer renderings of monsters are layered over the live stream of images). The image parameters may also affect certain of the objects appearing in the live steam of images generated by the camera.

In some examples, the image pair may be provided while the patient is wearing a virtual reality headset or virtual reality glasses. In some cases, the information content of the first image may be layered (e.g. an overlay of certain critters to avoid in the game, or powerups to collect in the game, etc.) over a live stream of images generated from a camera.

In some examples, one or more image parameters may affect objects appearing in the live stream of images generated from a camera (e.g. a filter, or changing the colour of certain trees perceived in the game such that they appear blue, where the patient would have to either select or avoid the blue trees, etc.)

The patient is then requested to perform a task while utilizing at least the information content of the image perceived by the weak eye at step 830. For instance, the task may be that of completing a video game, where information (e.g. objects, characters) perceivable only from the image presented to the weak eye is necessary to complete the game. In some examples, information presented in both images may be necessary to complete the game.

The patient's performance when completing the game may be recorded at step 840. The performance may be stored in memory of the apparatus 100. The performance may also be transmitted to a supervising physician (e.g. via a wired or wireless connection).

In accordance with the patient's observed or recorded performance, the image parameters of the image pair may be adjusted at step 850. The adjustment may take place on a timely basis (such as every week), where the program code, when executed by the processor, results in periodic adjustments of the image parameters as a function of the recorded results (e.g. the score obtained by the patient when performing the game). In some examples, the adjustments may also be performed by the supervising physician.

If the patient is successfully completing the task, the adjustment may be such that the difference in the image parameters is reduced. For instance, if the contrast results in the information content of the image presented to the weak eye being sharper than that of the image presented to the dominant eye, the adjustment may result in reducing the difference in contrast between the two perceived images. However, the difference in contrast between the two perceived images may be increased if the patient is having difficulty accomplishing the designated task.

Once the image parameters adjusted, steps 820 to 850 may be repeated with the adjusted image parameters. As such, as the training progresses and the difference in image parameters between the two perceived images of the image pairs is reduced as a function of the patient's capacity to accomplish the designated task, so will the patient improve the patient's diplopia and/or CID condition during the course of treatment.

Exemplary Study:

The following exemplary study demonstrates that the present apparatus (e.g. apparatus 100) may be used to treat and/or reduce the instances of diplopia in patients. The study shows that using the apparatus reduces the instances of diplopia in patients (e.g. in some cases, the patients may also use the technology to correct amblyopia). The subjects were treated and observed throughout the study to measure improvements of diplopia by reporting diplopia therethrough. The results presented herein relate to the improvement and, in some cases, disappearance of diplopia during the course of the study as the subjects were treated.

The study was designed to measure the treatment of amblyopia. However, it was shown, during the course of the study, that the use of the apparatus unexpectedly also improved diplopia amongst the patients having this condition. This is contrary to what was expected based on what was previously known in the art, as it was believed that the use of the apparatus would worsen diplopia and/or CID amongst the patients, and not improve these ocular conditions.

Study Design:

The subjects of the study met the following criteria:
Age 5 to <17 years
Amblyopia associated with anisometropia, strabismus (≤10Δ at near measured by PACT), or both
No amblyopia treatment (atropine, patching, Bangerter, vision therapy) in the past 2 weeks
Spectacles (if required) worn for at least 16 weeks, or demonstrated stability of visual acuity (<0.1 log MAR change by the same testing method measured on 2 exams at least 4 weeks apart)
Visual acuity in the amblyopic eye 20/40 to 20/200 inclusive (33 to 72 letters if E-ETDRS)
Visual acuity in the fellow eye 20/25 or better (≥78 letters if E-ETDRS)
Interocular difference ≥3 log MAR lines (≥15 letters if E-ETDRS)
No myopia greater than −6.00 D spherical equivalent in either eye
Ability to align the nonius cross on binocular game system. Heterotropia or heterophoria (total ocular deviation)≤10Δ by PACT at near is allowed, as long as the subject is able to align the nonius cross.
Demonstrate in-office ability to play Tetris game (on easy setting) under binocular conditions (with red-green glasses) by scoring at least one line

Subjects were randomly assigned (1:1) to either:
Binocular treatment group: binocular computer game play prescribed 1 hour per day 7 days a week, with a minimum of 4 days for children unable to play 7 days a week (treatment time can be split into shorter sessions totaling 1 hour)
Patching group: patching 2 hours per day for 7 days per week.

The sample sizes were as follows:
336 children aged 5 to <13 years (younger cohort)
166 children aged 13 to <17 years (older cohort)

The visit schedule is as follows (timed from randomization):
Enrollment exam
1 week phone call (7 to 13 days) to inquire about issues with the computer games (only for those assigned to binocular treatment and to be completed by site personnel)
4 weeks±1 week
8 weeks±1 week
12 weeks±1 week
16 weeks±1 week (primary outcome)

All subjects were seen at 4, 8, 12, and 16 weeks. Subjects achieving amblyopic-eye visual acuity equal to or better than the fellow-eye visual acuity (0 lines or more lines better, 0 letters or more better if E-ETDRS) and at least 20/25 (or >78 letters if E-ETDRS) visual acuity in both eyes is considered to have resolved and treatment is discontinued, although these subjects still returned for all remaining follow-up exams. If at a subsequent visit there is regression of amblyopia (2 log MAR lines or 10 letters), treatment is restarted.

At each follow-up visit, distance visual acuity is assessed in each eye using ATS-HOTV for children <7 years at enrollment and the E-ETDRS for children ≥7 years at enrollment. Stereoacuity is also assessed using the Randot Butterfly Stereoacuity test and Randot Preschool Stereoacuity test, history of diplopia, and ocular alignment by cover uncover test, simultaneous prism cover test (SPOT) (if deviation present), and prism and alternate cover test (PACT). A child and parental questionnaire to assess the impact of amblyopia treatment and diplopia is completed at 4 and 16 weeks.

Treatment and Follow-Up:

All subjects in the study played a Tetris-style game presented on an iPad while wearing red/green (anaglyph) glasses (over current spectacles, if applicable) with the green filter placed over the amblyopic eye. The subject is instructed to hold the iPad at his/her usual reading distance. Some boxes are only visible to the fellow eye viewing through the red lens, while other boxes are only visible to the amblyopic eye viewing through the green lens. Image contrast varies depending on depth of amblyopia to ensure stimulation of the amblyopic eye and binocular game play.

Contrast of Tetris shapes in the amblyopic eye (the weak eye) is at 100% throughout the study. Contrast of shapes seen by the fellow eye will begin at 20% at the start of the study and will increase or decrease automatically in 10% increments from the last contrast level (e.g., 20% to 22%) in a 24-hour period based on the subject's performance and duration of game play. As the ability of the subject to use the amblyopic eye or weak eye improves, game performance is expected to increase, and therefore the contrast setting in the fellow-eye will increase. The lower limit of fellow-eye contrast is set at 10%, which corresponds to the lower limit of the visible threshold for viewing objects on the screen. If the game settings remain at 10% for a period of 7 days, the game shows an alert for parents to contact their eye care provider.

Binocular Treatment Group

Subjects assigned to the binocular treatment group is prescribed a Tetris-style game to play for 1 hour per day, 7 days a week (with a minimum of 4 days a week for children unable to play 7 days a week) for 16 weeks. Parents of subjects are instructed that the 1 hour of daily treatment should be completed in a single 60-minute session, but if this is not possible for whatever reason, the treatment may have been divided into shorter sessions totaling 1 hour. The difficulty setting (easy, medium, or hard) is at the discretion of the child.

Patching Group

Subjects assigned to the patching group wear an adhesive patch over the fellow eye for 2 hours per day, 7 days per week for 16 weeks.

Compliance

Parents are asked to complete a compliance calendar by manually recording the number of minutes that the child played the game each day or how long the patch was worn. Calendars are reviewed by the investigator at each follow-up visit. The amount of time the game is played is also recorded automatically during game play by the iPad. These data are downloaded at the site during each follow-up visit when the iPad is brought to the study visit.

Phone Call for Those Assigned to Binocular Treatment

For those assigned to binocular treatment, site personnel call at 1 week (7 to 13 days) to confirm that there are no technical problems playing the binocular game and to address any questions.

Follow-Up Visit Schedule

The follow-up schedule is timed from randomization as follows:
4 weeks±1 week
8 weeks±1 week
12 weeks±1 week
16 weeks±1 week

Subjects achieving amblyopic-eye visual acuity equal to or better than the fellow-eye visual acuity (0 lines or more lines better, 0 letters or more better if E-ETDRS) and at least 20/25 (or >78 letters if E-ETDRS) visual acuity in both eyes are considered to have resolved and will discontinue treatment, although these subjects will still return for all remaining follow-up exams. If at a subsequent visit there is regression of amblyopia (2 log MAR lines or 10 letters), treatment is restarted.

Additional non-study visits can be performed at the discretion of the investigator.

Follow-Up Visit Testing Procedures

Subjects are at follow-up visits. Distance visual acuity and stereoacuity testing at these visits must be completed by a Masked Examiner. All procedures are performed with the subject's current refractive correction. If a subject currently wears spectacles but is not wearing them at the follow-up examination for whatever reason, testing must be performed in trial frames.

Prior to the Masked Examiner entering the room, subjects and parents are instructed not to discuss their treatment with the Masked Examiner.

The following procedures is performed in the following sequential order at each visit:

1. Impact of Amblyopia Treatment Questionnaire
The child and parent completed a short questionnaire to assess the impact of amblyopia treatment (to be completed only at the 4-week and 16-week visit)
For the parent, the questionnaire can be either self-administered or administered by the site staff; for the child, the questionnaire will be administered by site staff.
The questionnaire should be completed prior to the investigator's examination of the subject.
The questionnaire is meant for the child's parent or guardian who is responsible for administering the patching or overseeing binocular treatment. If the child is brought to the visit by an individual who is not involved in the treatment, this is indicated on the questionnaire, and the questionnaire is not completed.

2. Distance Visual Acuity Testing (Masked):
Monocular distance visual acuity testing will be performed in habitual refractive correction in each eye using the same visual acuity testing method that was used at enrollment, as described in the ATS Testing Procedures Manual.
Testing must be completed without cycloplegia.

3. Stereoacuity Testing (Masked):
Stereoacuity is tested in habitual current refractive correction using the Randot Butterfly test and Randot Preschool Stereoacuity test at near (⅓ meter).

4. Ocular Alignment Testing:
Ocular alignment is assessed in habitual refractive correction by the cover/uncover test, simultaneous prism and cover test (SPOT), and prism and alternate cover test (PACT) in primary gaze at distance (3 meters) and at near (⅓ meter) as outlined in the ATS Procedures Manual.

5. History of Diplopia

The child and parent(s) are specifically questioned regarding the presence and frequency of any diplopia since the last study visit using a standardized diplopia assessment (see ATS Miscellaneous Testing Procedures Manual).

Results with Respect to Diplopia:

Data was collected on the subjects with respect to diplopia based on observations and reports made by the subjects and/or the parents of the subjects during the course of the study. The patients and/or the parents of the patients were asked to report incidents of diplopia during the course of the study. It was observed that the patients who completed more gameplay during the course of the study had less of a chance to develop diplopia than the patients who performed less gameplay.

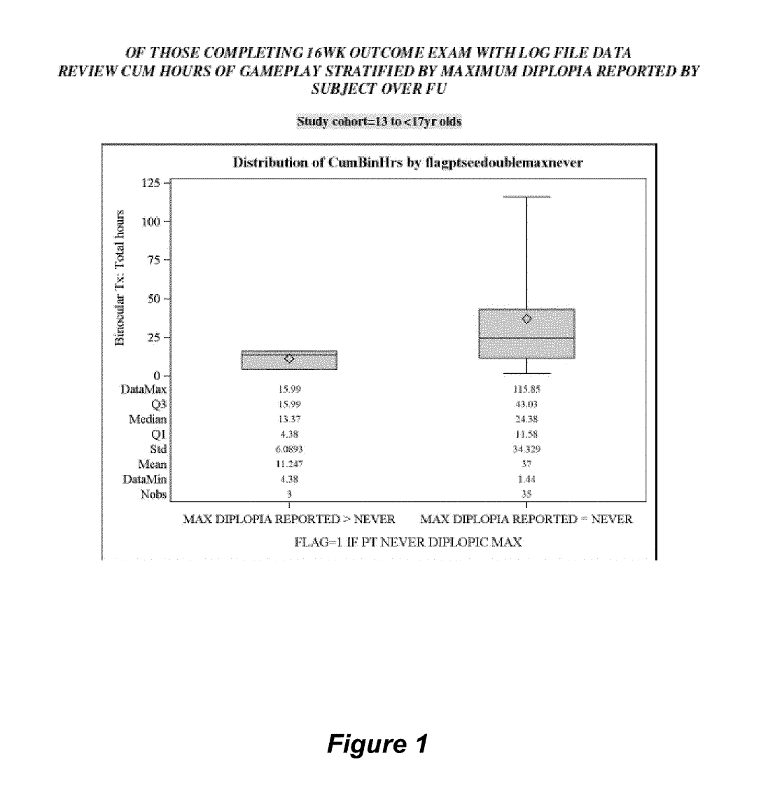

Table 1 relates to the instances of diplopia perceived by the subjects that are part of the study cohort between the ages of 13 to less than 17 years old during the course of treatment. The data is also presented in the graph FIG. 1, demonstrating that as the subjects performed more gameplay, so did the instances of diplopia reduce.

TABLE 1
diplopia perceived by the subjects that are part of the
study cohort between the ages of 13 to less than 17 years old.
Table of PtSeeDoubleOftenMax by TrtGroup

| PtSeeDoubleOftenMax(Particpant: | | | |
|---|---|---|---|
| Max Frequency of Diplopia) | TrtGroup(Treatment Group) | | |
| Frequency Col Pct | IPAD | PATCHING | Total |
| A) Never | 37 | 52 | 89 |
| | 92.50 | 86.67 | |
| B) Less than once a week | 1 | 1 | 2 |
| | 2.50 | 1.67 | |
| C) Once a week | 0 | 3 | 3 |
| | 0.00 | 5.00 | |
| D) Once a day | 2 | 3 | 5 |
| | 5.00 | 5.00 | |
| E) Up to 10 times a day | 0 | 1 | 1 |
| | 0.00 | 1.67 | |
| Total | 40 | 60 | 100 |

Diplopia was more recurrent amongst the subjects using the patch that those using the apparatus to perform gameplay.

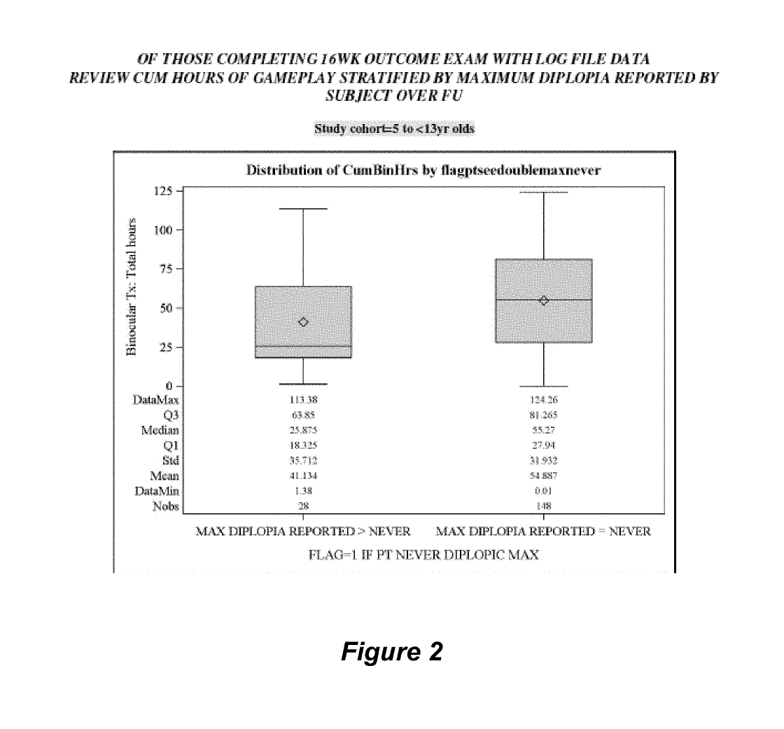

Table 2 relates to the instances of diplopia perceived by the subjects that are part of the study cohort between the ages of 5 to less than 13 years old during the course of treatment. The data is also presented in the graph of FIG. 2, demonstrating that as the subjects performed more gameplay, so did the instances of diplopia reduce.

TABLE 2
diplopia perceived by the subjects that are part of the study
cohort between the ages of 5 to less than 13 years old.
Table of PtSeeDoubleOftenMax by TrtGroup

| PtSeeDoubleOftenMax(Particpant: Max | | | |
|---|---|---|---|
| Frequency of Diplopia) | TrtGroup(Treatment Group) | | |
| Frequency Col Pct | IPAD | PATCHING | Total |
| A) Never | 157 | 172 | 329 |
| | 84.41 | 89.12 | |
| B) Less than once a week | 6 | 10 | 16 |
| | 3.23 | 5.18 | |
| C) Once a week | 10 | 4 | 14 |
| | 5.38 | 2.07 | |
| D) Once a day | 8 | 5 | 13 |
| | 4.30 | 2.59 | |
| E) Up to 10 times a day | 3 | 2 | 5 |
| | 1.61 | 1.04 | |
| F) >10 times a day | 1 | 0 | 1 |
| | 0.54 | 0.00 | |
| G) All the time | 1 | 0 | 1 |
| | 0.54 | 0.00 | |
| Total | 186 | 193 | 379 |

Frequency Missing = 6

Diplopia was more recurrent amongst the subjects between the ages of 5 to less than 13 years old using the patch that those using the apparatus to perform gameplay.

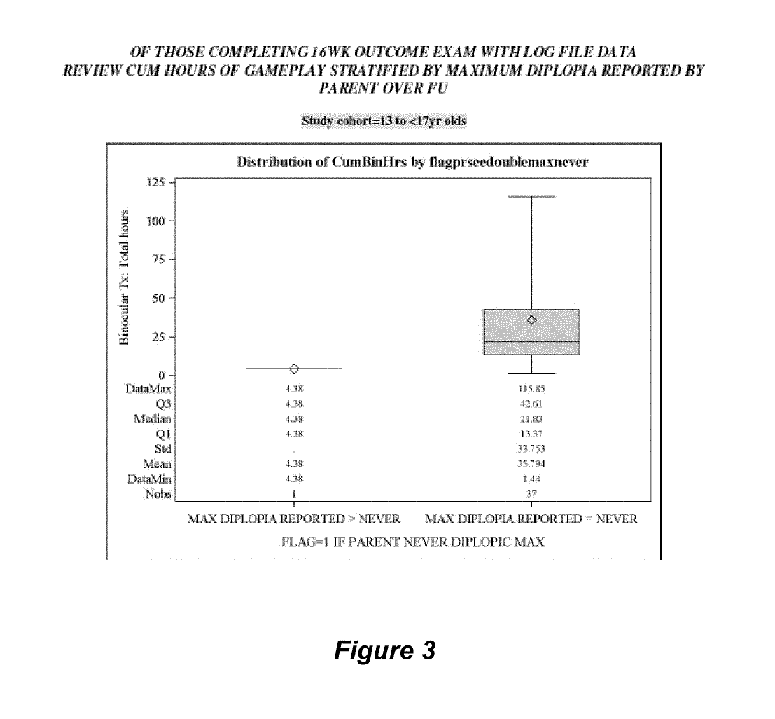

Table 3 relates to the instances of diplopia perceived by the parents of the subjects that are part of the study cohort between the ages of 13 to less than 17 years old during the course of treatment. The data is also presented in the graph FIG. 3, demonstrating that as the subjects performed more gameplay, so did the instances of diplopia reduce.

TABLE 3
diplopia perceived by the parents that are part of the study
cohort between the ages of 13 to less than 17 years old.
Table of PrSeeDoubleOftenMax by TrtGroup

| PrSeeDoubleOftenMax(Parent: | | | |
|---|---|---|---|
| Max Frequency of Diplopia) | TrtGroup(Treatment Group) | | |
| Frequency Col Pct | IPAD | PATCHING | Total |
| A) Never | 39 | 56 | 95 |
| | 97.50 | 93.33 | |
| B) Less than once a week | 0 | 2 | 2 |
| | 0.00 | 3.33 | |
| C) Once a week | 1 | 1 | 2 |
| | 2.50 | 1.67 | |
| D) Once a day | 0 | 1 | 1 |
| | 0.00 | 1.67 | |
| Total | 40 | 60 | 100 |

Diplopia was more recurrent amongst the subjects using the patch that those using the apparatus to perform gameplay, as observed by the parents.

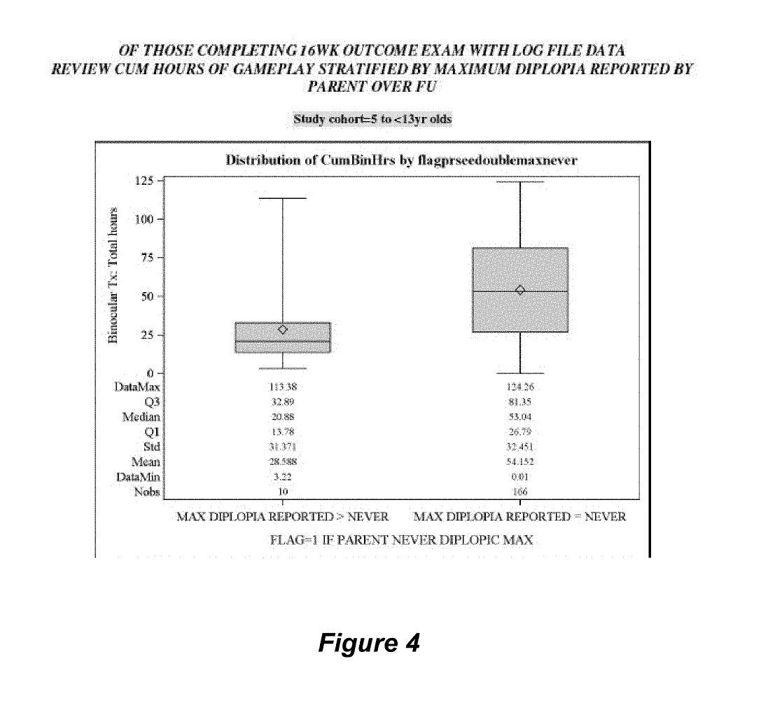

Table 4 relates to the instances of diplopia perceived by the parents of the subjects that are part of the study cohort between the ages of 5 to less than 13 years old during the course of treatment. The data is also presented in the graph of FIG. 4, demonstrating that as the subjects performed more gameplay, so did the instances of diplopia reduce.

TABLE 4
diplopia perceived by the parents that are part of the study
cohort between the ages of 5 to less than 13 years old.
Table of PrSeeDoubleOftenMax by TrtGroup

| PrSeeDoubleOftenMax(Parent: | | | |
|---|---|---|---|
| Max Frequency of Diplopia) | TrtGroup(Treatment Group) | | |
| Frequency Col Pct | IPAD | PATCHING | Total |
| A) Never | 176 | 187 | 363 |
| | 94.62 | 96.89 | |
| B) Less than once a week | 6 | 4 | 10 |
| | 3.23 | 2.07 | |
| C) Once a week | 2 | 1 | 3 |
| | 1.08 | 0.52 | |
| D) Once a day | 2 | 0 | 2 |
| | 1.08 | 0.00 | |
| E) Up to 10 times a day | 0 | 1 | 1 |
| | 0.00 | 0.52 | |
| Total | 186 | 193 | 379 |

Frequency Missing = 6

Diplopia was more recurrent amongst the subjects using the patch that those using the apparatus to perform gameplay as perceived by the parents.

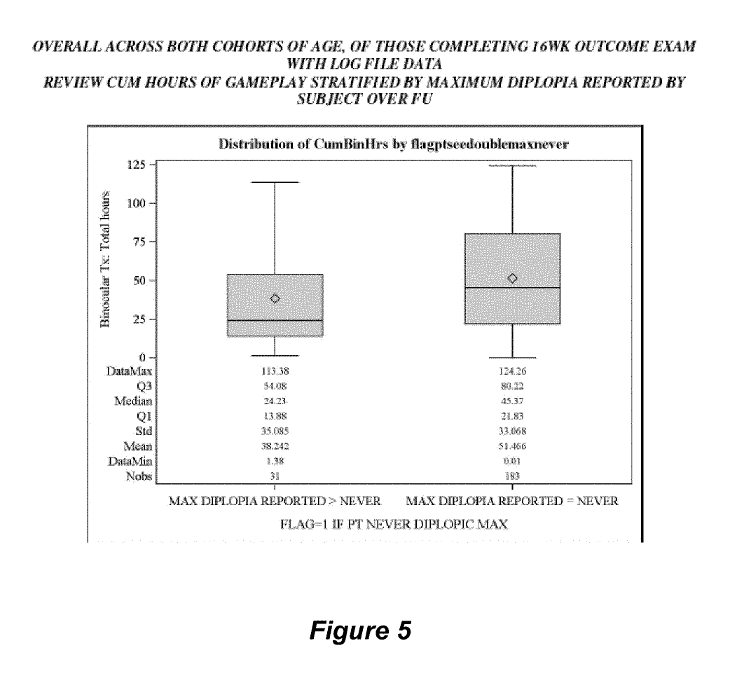

FIG. 5 illustrates the reports of diplopia as a function of gameplay over all age groups as perceived by the subjects.

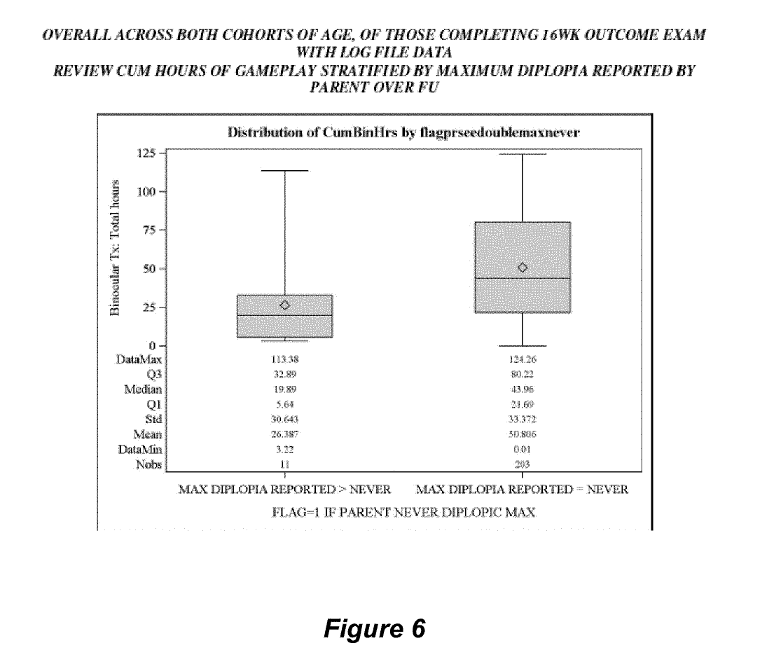

FIG. 6 illustrates the reports of diplopia as a function of gameplay over all age groups as perceived by the parents of the subjects.

As shown in FIGS. 1-6, the more the subjects played the games using an apparatus as described herein, lower were the chances that the patients developed diplopia. The patients that played more of the game prescribed by the treatment had less of a chance of developing diplopia than those that played less of the game as shown in FIGS. 1 through 6. Similarly, it will be understood that the patients who performed more gameplay would also experience less cases of CID, as a wandering eye leads to double vision, and less double-vision would result in the eye having less of a tendency to wander. It may be submitted that the performance of the gameplay strengthens the extraocular muscles, which may reduce the instances of diplopia and CID.

These results are unexpected as it has been postulated by persons skilled in the art that use of such an apparatus as apparatus 100 and/or as described in the present study, would in fact cause diplopia. However, for example, as observed in the present study, it has been demonstrated that such an apparatus reduces the symptoms of diplopia and/or CID, and may in fact be used to treat either one or both of these conditions.

Representative, non-limiting examples of the present invention were described above in detail with reference to the attached drawings. This detailed description is merely intended to teach a person of skill in the art further details for practicing preferred aspects of the present teachings and is not intended to limit the scope of the invention. Furthermore, each of the additional features and teachings disclosed above and below may be utilized separately or in conjunction with other features and teachings to provide useful apparatuses and methods of treatment using the same.

Moreover, combinations of features and steps disclosed in the above detailed description, as well as in the experimental examples, may not be necessary to practice the invention in the broadest sense, and are instead taught merely to particularly describe representative examples of the invention. Furthermore, various features of the above-described representative examples, as well as the various independent and dependent claims below, may be combined in ways that are not specifically and explicitly enumerated in order to provide additional useful embodiments of the present teachings.

All features disclosed in the description and/or the claims are intended to be disclosed separately and independently from each other for the purpose of original written disclosure, as well as for the purpose of restricting the claimed subject matter, independent of the compositions of the features in the embodiments and/or the claims.

What is claimed is:

1. A method of treating diplopia or convergence insufficiency disorder in a patient comprising:
providing a patient having a condition of diplopia or convergence insufficiency disorder with an image pair configured to present a first image to a first weaker eye of said patient and a second image to a second dominant eye of said patient, wherein information content of said first image perceivable by said first eye is different from information content of said second image perceivable by said second eye, and wherein the perceptibility of said information content of said first image is augmented in comparison to the perceptibility of said information content of said second image as a result of a difference in at least one image parameter of said first image and said second image;
obtaining performance information of said patient when said patient performs a task requiring the perceiving of at least said information content of said first image; and
adjusting, based on said performance information, when said performance information is indicative of an improvement of said patient's diplopia or convergence insufficiency disorder symptoms, said at least one image parameter such that said difference in perceptibility of said information content of said first image and said information content of said second image is reduced.

2. The method as defined in claim 1, wherein said image pair is generated from a single image source configured to be used with anaglyphic glasses, wherein said patient wearing said anaglyphic glasses results in the presenting of said first image to said first weaker eye of said patient and said second image to said second dominant eye of said patient.

3. The method as defined in claim 1, wherein said image pair comprises a first image source for generating said first image presented to said first eye and a second image source for generating said second image presented to said second eye.

4. The method as defined in claim 1, wherein said image pair is generated from an image source configured to generate an image stream.

5. The method as defined in claim 1, wherein said at least one image parameter is the number of objects appearing in said first image and the number of objects appearing in said second image.

6. The method as defined in claim 1, wherein said at least parameter is the contrast of said first image and said second image.

7. The method as defined in claim 1, wherein said difference in perceptibility affects only a portion of at least one of said first image and said second image.

8. The method as defined in claim 1, wherein said task is established within the context of a video game.

9. The method as defined in claim 1, wherein said image pair is provided while said patient is wearing an augmented reality headset.

10. The method as defined in claim 9, wherein said information content of said first image is layered over a live stream of images generated from a camera.

11. The method as defined in claim 9, wherein said at least one image parameter affects objects appearing in the live stream of images generated from a camera.

12. The method as defined in claim 1, wherein said image pair is provided while said patient is wearing a virtual reality headset or virtual reality glasses.

13. The method as defined in claim 12, wherein said information content of said first image is layered over a live stream of images generated from a camera.

14. The method as defined in claim 12, wherein said at least one image parameter affects objects appearing in the live stream of images generated from a camera.

15. The method as defined in claim 1, wherein said patient has diplopia.

16. The method as defined in claim 1, wherein said patient has convergence insufficiency disorder.
